(19) United States
(12) Patent Application Publication
(10) Pub. No.: US 2007/0083175 A1
(43) Pub. Date: Apr. 12, 2007

(54) Transparent/translucent absorbent composites and articles

(75) Inventors: Timothy James Van Himbergen, Appleton, WI (US); Renee Sue Kole, Oshkosh, WI (US); Dave Allen Soerens, Neenah, WI (US); David Arthur Fell, Neenah, WI (US); Ligia de los Angeles Rivera, Appleton, WI (US); Hoa La Wilhelm, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/247,734

(22) Filed: Oct. 11, 2005

(51) Int. Cl. A61F 13/15

(52) U.S. Cl. 604/367; 604/378

(57) ABSTRACT

The present invention provides a translucent absorbent composite having a substrate with a light transmittance of at least about 60% and an absorbent material applied to the substrate. The absorbent layer contains an absorbent prepared from a water soluble ionic polymer having about 15 to about 99.9% by mass monoethylenically unsaturated polymer units having at least one functional group and about 0.1 to about 20% by mass ester units selected from the group consisting of acrylate and methacrylate ester units having an alkoxysilane functionality, wherein the absorbent composite has a light transmittance of at least 45%. Also provided are absorbent articles containing the absorbent composite.

### FIELD OF THE INVENTION

The present invention relates generally to translucent absorbent composites having a high percentage of light transmission and translucent absorbent articles prepared from the absorbent composites also having a high percentage of light transmission.

### BACKGROUND OF THE INVENTION

Absorbent articles have been used in a wide variety of uses, including in hygiene and health related applications. Currently, absorbent articles used in hygiene related applications include, for example, pantiliners, sanitary napkins, incontinence pads, and incontinence garments. Examples of absorbent articles used in heath related applications include, for example, bandages, among many other items. Absorbent articles have been used in other applications, such as bed pads and furniture pads. In each case, absorbent articles are typically white or are colored to a desired color depending on the manufacturer and/or the intended end use. Color is generally imparted with pigments, fillers or dyes which are added to the raw materials used to make the absorbent article. Color has been historically used in absorbent articles to communicate a hygienic condition of the article prior to use. Typically, a white color has been used as the predominate color of absorbent articles to convey that the absorbent articles are of a hygienic condition.

Current fashion trends have resulted in undergarments having colors other than the traditional white. In addition, these current fashion trends have yielded outer clothing which has a certain “see-through” quality, such that the color of the undergarments can be easily recognized through the outer clothing. By having absorbent articles, such as pantiliners and sanitary napkins, with a white color and the undergarments of a different color, the white color may make the absorbent article or the contour of the absorbent article visible through the undergarment and outer clothing, resulting in a lack of discretion for the user of the absorbent article. In addition, coloring the absorbent article to match the coloring of the undergarments would be a possible option to provide discretion. However, preparing absorbent articles that will match or nearly match the color of the wide variety of undergarments currently being manufactured by the clothing industry would be a nearly impossible task for a manufacturer of absorbent articles. For each product line, the manufacture of absorbent articles would need to provide various colors of absorbent articles. In addition, having many different colors would result in many different product codes that a retailer would need to stock on its shelves. With shelf space being a premium in today's retail market, providing sufficient shelf space for a given manufacturer to display its products would or could require more than twice the shelf space currently available. Furthermore, simple changes in a dye lot for the absorbent articles or the undergarments could result in absorbent articles that would not match or nearly match the undergarments.

Currently available absorbent articles typically do not blend in with the surrounding use environments; thereby providing little, if any, discretion to the user due to color of the articles. While the absorbent articles may blend in with environments that have the same or very close color hue to that of the absorbent article, the absorbent articles do not blend in with a use environment that is of a different color hue. For example, in the case of a pantiliner, if the pantiliner is white and the undergarment in which the pantiliner is placed is a different color, for example, black, the pantiliner may be readily visible in the undergarment. This problem can be exasperated by placing the absorbent article in a use environment which has a pattern, such as in the case of furniture, bed sheets and undergarments.

Attempts have been made in the art to make absorbent articles less noticeable to non-users. For example, BAND-AID® bandages, available from Johnson & Johnson, offers a clear or transparent attachment strip bandage. In this product, the absorbent area of the bandage is clearly distinguished from the surrounding attachment strip, making the absorbent area noticeable to non-users. Other attempts have been made in the art to provide discretion to absorbent articles, including making wings or flaps on feminine care products translucent.

There is a need in the art for absorbent articles which are transparent or translucent so that the absorbent articles will blend in with the use environments and can be discretely used by a user without others readily determining that the user is using an absorbent article. In addition, there is a need for transparent or translucent absorbent articles which are also effective in absorbing fluids.

### SUMMARY OF THE INVENTION

Generally stated, the present invention provides a translucent or nearly transparent absorbent composite. The absorbent composite of the present invention may be used in an absorbent article, providing an absorbent article that will blend with the use environment of the absorbent article, and will provide sufficient capacity for a given absorption task. In the present invention, the absorbent composite has a substrate and an absorbent material applied to the substrate. The absorbent material contains an absorbent prepared from a water soluble ionic polymer having about 15 to about 99.9% by mass monoethylenically unsaturated polymer units having at least one functional group, about 0.1 to about 20% by mass ester units selected from the group consisting of acrylate and methacrylate ester units having an alkoxysilane functionality. The absorbent composite in the absorbent area has a light transmittance of at least 45%. In one embodiment of the present invention, the absorbent composite may have a light transmission of at least 60%.

In another embodiment of the present invention, the absorbent composite has at least one additional layer. In this embodiment of the present invention, an additional layer is adjacent the substrate with the absorbent material applied to the substrate. The additional layer may be selected to impart different properties to the absorbent composite including as a fluid intake layer or as a fluid impermeable layer. More than one additional layer may be present in the absorbent composite of the present invention. In an additional aspect of this embodiment of the present invention, the additional layer may have a light transmittance of at least 60%.

In an additional embodiment of the present invention, two additional layers are present in the absorbent composite. When two additional layers are present in the composite, the substrate and absorbent material applied thereto may be a top or intermediate layer of the composite. In this regard, one configuration of the composite has the substrate with the absorbent material applied thereto positioned between the first and second additional layers. A further embodiment of this aspect of the invention, each of the first and second layers may have a light transmittance of at least 60%.

In yet another embodiment of the present invention, provided is a translucent absorbent composite having a backing, and an absorbent layer, where the absorbent layer is positioned adjacent the backing layer, and the absorbent layer contains a substrate and an absorbent material applied to the substrate. The absorbent material is an absorbent prepared from a water soluble ionic polymer having about 15 to about 99.9% by mass monoethylenically unsaturated polymer units having at least one functional group, about 0.1 to about 20% by mass ester units selected from the group consisting of acrylate and methacrylate ester units having an alkoxysilane functionality, wherein the absorbent layer has a light transmittance of at least 45%. The backing layer has a light transmittance of at least 60%, and the overall absorbent composite has a minimum light transmittance of at least 45%. In a further aspect of this embodiment, the absorbent composite may have a perimeter region and a central region. The backing layer is present in both the perimeter region and the central region, and the absorbent layer is positioned adjacent the backing layer and is only present in the central region of the absorbent composite. In yet a further aspect of this embodiment of the present invention, the perimeter region may have a light transmittance of at least 60% and the central region may have a light transmittance of at least 45%. The absorbent composite of this aspect of the present invention may also have a liner layer, where the absorbent layer is positioned between the liner layer and the backing layer. The liner layer also has a light transmittance of at least 60%.

Also provided by the present invention is an absorbent article prepared from the absorbent composite. In this embodiment, an absorbent article of the present invention has a backing layer, an absorbent layer and a bodyside liner. The absorbent layer is positioned between the bodyside liner and the backing layer, the absorbent layer has a substrate and an absorbent material applied to the substrate. The absorbent material applied to the substrate is prepared from a water soluble ionic polymer having about 15 to about 99.9% by mass monoethylenically unsaturated polymer units having at least one functional group, and about 0.1 to about 20% by mass ester units selected from the group consisting of acrylate and methacrylate ester units having an alkoxysilane functionality. The absorbent layer of the absorbent article has a light transmittance of at least 45% and the backing layer and the bodyside liner each have a light transmittance of at least 60%. The absorbent article of this aspect of the present invention has a minimum light transmittance of at least 45%.

In yet another embodiment of the present invention, the absorbent article has a perimeter region and a central region. The backing layer and the bodyside liner are each present in both the perimeter region and the central region, and the absorbent layer is positioned between the backing layer and the bodyside liner layer. The absorbent layer is only present in the central region of the absorbent article. In yet a further aspect of this embodiment of the present invention, the perimeter region may have a light transmittance of at least 60% and the central region may have a light transmittance of at least 45%. In another aspect of this embodiment of the present invention, the perimeter region of the absorbent article has a light transmittance of at least 80% and the central region of the absorbent article has a light transmittance of between about 65% and about 79%. Absorbent articles of the present invention include, for example, sanitary napkin, an incontinence pad, a pantiliner, a bandage, a bed pad or a furniture pad.

In a further embodiment of the present invention, the present invention also provides an absorbent article comprising a body contacting surface, a surface opposed the body contacting surface, an absorbent core position between the body contacting surface and the surface opposed the body contacting surface, longitudinal edges extending along an edge of absorbent core and flaps. The flaps extend from the longitudinal edges of the absorbent article and the flaps contain an absorbent material, which is capable of absorbing fluids. The flaps have a light transmittance of at least 45%.

### BRIEF DESCRIPTION OF THE DRAWINGS

FIGS. 1A and 1B show a cross-section of an absorbent composite of the present invention having a substrate layer and an absorbent layer.

FIG. 1C shows a cross-section of an absorbent composite of the present invention having a substrate layer impregnated with an absorbent material.

FIGS. 2A and 2B show a cross-section of an absorbent composite of the present invention having a substrate layer impregnated with the absorbent material and an additional layer.

FIGS. 2C and 2D show a cross-section of an absorbent composite of the present invention having a substrate layer, an absorbent layer and an additional layer.

FIGS. 3A and 3B show a cross-section of an absorbent composite of the present invention having a substrate layer impregnated with the absorbent material and two additional layers.

FIG. 4 shows a top view cut-away view of an absorbent article of the present invention.

FIG. 5 shows a cross-section of the absorbent article of the present invention.

FIG. 6A shows a cross-section side view of an absorbent bandage of the present invention.

FIG. 6B shows a top perspective view of an absorbent bandage of the present invention.

FIG. 7 shows a top perspective view of an absorbent bed or furniture liner of the present invention.

### DEFINITIONS

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

It should be understood that the term “personal care product” or “personal care article” as used herein refers to any article used to control bodily fluids, and includes “absorbent products,” which refers to any article configured to absorb and retain bodily exudates, including urine, bowel movements, blood and menses, and includes such a product in a packaged and unpackaged configuration. As such, “personal care articles” as used herein, includes, without limitation, diapers, child toilet training pants, adult incontinence garments, male incontinence products, tampons, vaginal suppositories, pantiliners, pads, sanitary napkins, tissues, wipes, etc. For example, personal care articles include, without limitation, Poise® feminine care articles, including pantiliners and pads, and Kotex® feminine care articles, including sanitary napkins, tampons and liners, all available from Kimberly-Clark Corporation, Neenah, Wis.

As used herein, the term “connected” is intended to mean that two or more members are directly or indirectly connected to one another. When two or more members are directly connected to one another, it is meant that the two members are in direct contact with one another, without an intervening member or structure. When two or more members are indirectly connected to one another, it is meant that the two members are not in direct contact with one another, and may have an intervening member or structure between the two or more members connected to one another.

“Binder” includes materials which are capable of attaching themselves to a substrate or are capable of attaching other substances to a substrate.

“Fluid” refers to a substance in the form of a liquid or gas at room temperature and atmospheric pressure.

“Knife over roll coating” refers to a process in which a knife is positioned, with a specified gap, above a substrate that is moving beneath the knife on a moving roll. In this manner, the knife spreads a specified thickness of coating material onto the substrate.

“Slot coating” refers to a process in which a slot die provides a thin, uniform coating on a substrate to be coated. In slot coating, the coating can be placed using a open gap in which the substrate to be coated is passed under the slot die, or a closed gap, in which the slot die is aligned with a coating roll, such that there is a narrow gap or nip between the roller and slot die. The substrate to be coated is passed between the coating roll and the slot die.

“Layer” when used in the singular can have the dual meaning of a single element or a plurality of elements.

“Modifying agent” refers to a substance that may be added to a composition to modify the physical properties of the composition, such as the color or texture of the composition.

“Nonwoven” or “nonwoven web” refers to materials and webs or material having a structure of fibers or filaments which are interlaid, but not in an identifiable manner as in a knitted fabric. The terms “fiber” and “filament” are used interchangeably. Nonwoven fabrics or webs have been formed from many processes such as, for example, meltblowing processes, spunbonding processes, air laying processes, and bonded carded web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

“Personal care absorbent product” includes diapers, diaper pants, training pants, absorbent underpants, adult incontinence products, feminine hygiene products, and the like.

“Roll printing” or “roll coating” refers to a process in which the application of a deposited material, generally as a paste, onto a substrate is carried out by transferring the deposited material from a roll onto the substrate in a more or less uniform layer using one or more rolls, which may be engraved, or a pool cylinder. A doctor blade is used to scrape any excess deposited material from the rolls or substrate. The doctor blade may be flat or have a patterned edge such as slots or ridges.

“Rotary screen printing” or “rotary screen coating”, refers to a process that is a combination of roll printing or coating and screen printing or coating.

“Screen printing” or “screen coating” refers to a method of applying a deposited material by forcing the material to be deposited through a screen that may have uniform openings or patterned openings.

“Superabsorbent” refers to a water-swellable, water-insoluble organic or inorganic material capable, under the most favorable conditions, of absorbing at least about 10 times its weight and, more desirably, at least about 15 times its weight in an aqueous solution containing 0.9 weight percent sodium chloride. The superabsorbent materials can be natural, synthetic, and modified natural polymers and materials. In addition, the superabsorbent materials can be inorganic materials, such as silica gels, or organic compounds such as cross-linked polymers. A material is “absorbent” if it absorbs at least five times its weight of the aqueous solution under these conditions.

“Unit” or “polymer unit” refers to a monomer or polymer portion of a copolymer molecule or blend component that includes a different molecular structure, compared to another portion of the copolymer or blend.

As used herein, the term “support layer” refers to a layer of the absorbent article in which the absorbent layer is formed.

As used herein, the term “liquid impermeable” means a layer that is substantially impermeable or otherwise impermeable to liquids intended to be absorbed by the absorbent article.

As used herein, the term “liquid permeable” means a layer that is operatively permeable to liquids intended to be absorbed by the absorbent article.

### DETAILED DESCRIPTION OF THE INVENTION

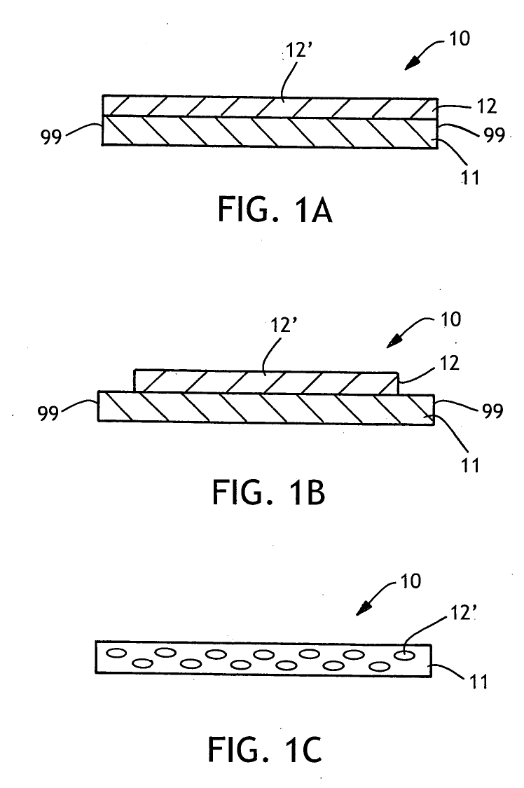

The absorbent composite of the present invention contains a substrate and an absorbent material applied to the substrate. The absorbent material may be a layer on the substrate, as is shown in FIGS. 1A and 1B, which are described in more detail below, or the absorbent material may be impregnated into the substrate, as is shown in FIG. 1C. To gain a better understanding of the present invention, attention is directed to FIG. 1A, which shows a cross-section of the absorbent composite of the present invention. In FIG. 1A, the absorbent composite 10 has a substrate or support layer 11 and an absorbent layer 12. In one embodiment of the present invention, the absorbent layer may be formed on a surface of the substrate from an absorbent material 12′. As will be explained in more detail below, the absorbent material is prepared from an absorbent binder composition. As is shown in FIG. 1A, the absorbent layer 12 is coextensive with the substrate 11. However, in the present invention, it is not necessary that the absorbent layer 12 is coextensive with the substrate layer 11. That is, the absorbent layer 12 does not completely cover the substrate 11 to the outer edges 99 of the substrate. In an alternative embodiment of the present invention shown in FIG. 1B, the absorbent layer 12 containing the absorbent material 12′ is not coextensive with the substrate 11, covering only a portion of the substrate 11 short of the outer edges 99 of the substrate 11. In another alternative embodiment of the present invention, the absorbent material may be placed within the substrate or impregnated within the substrate. This is shown in FIG. 1C, where the absorbent material 12′ is placed within the substrate 11. In order for the absorbent material to be impregnated or otherwise placed within the substrate 11, the substrate should be prepared from a material which contains interstitial spaces that allow the absorbent material to penetrate the surface of the substrate and allow the absorbent material 12′ to be within interstitial spaces within the substrate 11. Whether the absorbent is a layer on the substrate or placed within the substrate, the substrate acts as a support layer, supporting the absorbent material and the absorbent layer is generally prepared on the substrate. It is noted that the absorbent material 12′ appears to be shown in FIG. 1C a discrete phase or discrete particles; however the intent is to show that the absorbent material 12′ is impregnated within the substrate 11. That is the absorbent material could be a continuous phase within the substrate.

In the present invention, the substrate and the absorbent material combined have a light transmittance of at least 45%. In the present invention, light transmittance is measured by using a Gardner Haze Guard Plus Model 4725. To measure the light transmittance, a flat sample of the material to be tested was placed in a round holder having approximately a 60 mm diameter. Measurements are taken by placing the flat sample in a measuring port. The haze port is used for measuring light transmittance. A series of five samples are measured and the average value of the five samples is the light transmittance. Haze and clarity may also be measured using the Gardner Haze Guard Plus. Haze, clarity and light transmission are measured by ASTM D-1003. In an embodiment of the present invention, the light transmittance of the absorbent composite in an area containing the absorbent material is generally greater than 50% and is desirably at least 60%. Typically the light transmittance is in the desired range of 60-79%.

The substrate of the absorbent composite can be a wide variety of materials. The support layer can be liquid permeable or liquid impermeable. The support layer can be a film, a nonwoven web knitted fabric or a woven fabric, or a laminate of one or more of these materials. The only requirements for the substrate layer is that the support layer has a light transmittance of at least 45%, and the support has sufficient integrity so that the absorbent material may be placed on the substrate layer, or in the case of substrates with interstitial spaces, such as knitted fabrics, woven fabrics and nonwoven webs, or laminates containing these substrate materials, can be impregnated with the absorbent material. In addition, the substrate should have sufficient flexibility so the absorbent can be used in flexible absorbent personal care articles. Particular examples of substrates include, polyolefin films, spunbond nonwoven webs and laminates of polyolefin films and spunbond nonwoven webs, bonded-carded webs, bonded-airlaid webs, coform, and woven fabrics such as cotton and wool cloth.

The light transmittance of the substrate can be affected in many different ways. For example, the addition of coloring agents, such as dyes, pigments, fillers and other similar materials in the raw materials used to make the substrate may reduce the light transmittance of the substrate formed from the raw materials. One way to improve the light transmittance of the substrate is to reduce the amount or even eliminate coloring agents, pigments, fillers and other materials which may cause a reduction of light transmittance from the raw materials used to make the substrate. In one embodiment of the present invention, the substrate contains less than about 2% fillers, pigments or coloring agents which can reduce the light transmittance of the substrate. Desirably, the substrate contains less than about 1% by weight of fillers, pigments or coloring agents which can reduce the light transmittance of the substrate. Most desirably, the substrate is substantially free of coloring agents, pigments, fillers and other similar materials which may reduce the light transmittance of the substrate.

Other ways to improve or keep the light transmittance of the substrate in the range described above is to keep the basis weight and thickness of the substrate to a minimum. While keeping the thickness and basis weight to a minimum, care should be taken so the substrate has sufficient strength. In the present invention, when the substrate is a film material, the film should have a thickness of less than about 1.0 mm (broad thickness). Desirably, the thickness of the film is less than about 0.5 mm and generally greater than about 0.01 mm. Most desirably, the thickness of the film should be between about 0.02 mm and 0.25 mm. As the thickness of the film increases, the light transmittance of the film may be reduced. On the other hand, if the thickness of the film is less that about 0.01 mm, the substrate may be damaged during formation of the absorbent composite or during use of the absorbent composite, unless the film is reinforced in some manner, for example, laminating the film to a nonwoven web. When the substrate is a nonwoven web, generally the basis weight should be kept below about 100 gsm; however, the basis weight is only limited by the overall light transmission of the absorbent composite. Therefore, the nonwoven substrate could have a basis weight in excess of 100 gsm. Desirably, the basis weight of the nonwoven web should be between 7 gsm and 60 gsm. Most desirably, the basis weight of the nonwoven web should be between 10 gsm and 40 gsm. Generally, if the basis weight is above 100 gsm, the nonwoven web will tend to have a lower light transmittance. If the basis weight is below about 7 gsm, the nonwoven web will tend to have insufficient strength to support the absorbent material.

In the present invention, the absorbent material may be prepared from an absorbent binder composition. The absorbent binder composition is placed directly on the substrate and is directly joined to the substrate, without the addition of adhesives, thereby forming a layer on the substrate. In the alternative, the absorbent material will penetrate the substrate and will be impregnated into the support substrate. The absorbent binder composition may be applied to the substrate using any suitable application process, including knife over roll coating, or roll coating, either in a continuous coverage or a patterned coverage. Printing applications or other suitable application techniques, including gravure printing, screen, and jet printing. The absorbent binder composition may also be applied to the substrate using a spray application. The actual method of application of the absorbent binder to the substrate is not critical to the present invention. Once placed on the substrate, the absorbent binder composition is crosslinked, forming an absorbent coating on the substrate or forming a crosslihked absorbent material impregnated within the substrate.

The absorbent binder composition includes about 15 to about 99.8% by mass of monoethylenically unsaturated polymer units, suitably about 25 to about 90% by mass, particularly about 30 to about 79% by mass, or about 50 to about 70% by mass. Suitable monoethylenically unsaturated polymer units include without limitation monoethylenically unsaturated carboxylic acid units and salts thereof, monoethylenically unsaturated sulphonic acid units and salts thereof, and monoethylenically unsaturated phosphonic acid units and salts thereof. Suitable monoethylenically unsaturated monomers that can be used to form the monoethylenically unsaturated polymer units include without limitation:

a) Carboxyl group-containing monomers including monoethylenically unsaturated mono or poly-carboxylic acids, such as (meth)acrylic acid (meaning acrylic acid or methacrylic acid; similar notations are used hereinafter), maleic acid, fumaric acid, crotonic acid, sorbic acid, itaconic acid, and cinnamic acid; b) Carboxylic acid anhydride group-containing monomers, including monoethylenically unsaturated polycarboxylic acid anhydrides (such as maleic anhydride);

c) Carboxylic acid salt group-containing monomers including water-soluble salts (alkali metal salts, ammonium salts, amine salts, etc.) of monoethylenically unsaturated mono- or poly-carboxylic acids (such as sodium (meth)acrylate, trimethylamine (meth)acrylate, triethanolamine (meth)acrylate), sodium maleate, methylamine maleate;

d) Sulfonic acid group-containing monomers, including aliphatic or aromatic vinyl sulfonic acids (such as vinylsulfonic acid, allyl sulfonic acid, vinyltoluenesulfonic acid, stryrene sulfonic acid), (meth)acrylic sulfonic acids [such as sulfopropyl (meth)acrylate, 2-hydroxy-3-(meth)acryloxy propyl sulfonic acid];

e) Sulfonic acid salt group-containing monomers, including alkali metal salts, ammonium salts, amine salts of sulfonic acid group containing monomers as mentioned above; and/or

f) Amide group-containing monomers, including vinylformamide, (meth)acrylamide, N-alkyl (meth)acrylamides (such as N-methylacrylamide, N-hexylacrylamide), N,N-dialkyl (meth)acryl amides (such as N,N-dimethylacrylamide, N,N-di-n-propylacrylamide), N-hydroxyalkyl (meth)acrylamides [such as N-methylol (meth)acrylamide, N-hydroxyethyl (meth)acrylamide], N,N-dihydroxyalkyl (meth)acrylamides [such as N,N-dihydroxyethyl (meth)acrylamide], 3-acrylamidopropyl trimethyl ammonium chloride, vinyl lactams (such as N-vinylpyrrolidone).

The absorbent binder composition also includes about 0.1 to about 20% by mass of polyacrylate ester units, such as acrylate and/or methacrylate ester units, that include an alkoxysilane functionality. The acrylate and/or methacrylate ester units are copolymerized with the monoethylenically unsaturated monomer units. In particular, the absorbent binder composition may include about 0.5 to about 15% by mass of the acrylate and/or methacrylate ester units, for instance about 1.0 to about 10% by mass, for instance about 1.5 to about 5.5% by mass.

The alkoxysilane functionality is a functional group or moiety that reacts with water to form a silanol group. One suitable alkoxysilane group is a trialkoxy silane group having the following structure:

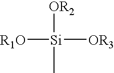

wherein R₁, R₂ and R₃ are alkyl groups independently having from 1 to 6 carbon atoms. Dialkoxysilane groups having the following formula may also be used: 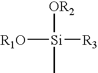
wherein R₁, R₂ and R₃ are alkyl groups independently having from 1 to 6 carbon atoms.

The term “monomer(s)” as used herein includes monomers, oligomers, polymers, mixtures of monomers, oligomers and/or polymers, and any other reactive chemical species which is capable of co-polymerization with monoethylenically unsaturated carboxylic, sulphonic or phosphoric acid or salts thereof. Ethylenically unsaturated monomers containing a trialkoxy silane functional group are appropriate for this invention and are desired. Suitable ethylenically unsaturated monomers include acrylates and methacrylates. A particularly ethylenically unsaturated monomer containing a trialkoxy silane functional group is methacryloxypropyl trimethoxy silane, commercially available from Dow Corning, having offices in Midland, Mich., under the trade designation Z-6030 Silane. Other suitable ethylenically unsaturated monomers containing a trialkoxy silane functional group include, but are not limited to, methacryloxyethyl trimethoxy silane, methacryloxypropyl triethoxy silane, methacryloxypropyl tripropoxy silane, acryloxypropylmethyl dimethoxy silane, 3-acryloxypropyl trimethoxy silane, 3-methacryloxypropylmethyl diethoxy silane, 3-methacryloxypropylmethyl dimethoxy silane, and 3-methacryloxypropyl tris(methoxyethoxy) silane. However, it is contemplated that a wide range of vinyl and acrylic monomers having trialkoxy silane functional groups or a moiety that reacts easily with water to form a silanol group, such as a chlorosilane or an acetoxysilane, provide the desired effects are effective monomers for copolymerization in accordance with the present invention.

In addition to monomers capable of co-polymerization that contain a trialkoxy silane functional group, it is also feasible to use a monomer capable of co-polymerization that can subsequently be reacted with a compound containing a trialkoxy silane functional group or a moiety that reacts with water to form a silanol group. Such a monomer may contain, but is not limited to, an amine or an alcohol. An amine group incorporated into the co-polymer may subsequently be reacted with, for example, but not limited to, (3-chloropropyl) trimethoxysilane. An alcohol group incorporated into the co-polymer may subsequently be reacted with, for example, but not limited to, tetramethoxysilane.

The absorbent binder composition may also include zero to about 75% by mass polyolefin glycol and/or polyolefin oxide units, suitably about 0.1 % to about 75% by mass, particularly about 5% to about 50% by mass particularly about 5% to about 40% by mass, particularly about 5% to about 30% by mass, particularly about 5% to about 20% by mass. The polyolefin glycol or oxide may be a glycol or oxide of an olefin polymer having about 2 to about 4 carbon atoms. Polyethylene glycol, polyethylene oxide, polypropylene glycol and polypropylene oxide are examples of suitable polymer units. The polyolefin glycol and/or polyolefin oxide, may include on average about 30 to about 15,000 glycol and/or oxide units per molecule. The weight average molecular weight of polyolefin glycol units may range from about 200 to about 8000. When polyolefin oxide units are employed, they may have a weight average molecular weight of about 100,000 to about 600,000.

Polyolefin glycols and polyolefin oxides are commercially available, and are common. To prepare the absorbent binder composition, a pre-formed polyolefin glycol and/or oxide may be dissolved or dispersed in a reaction vessel which includes an aqueous solvent or carrier, an organic solvent or carrier such as ethanol, or a miscible combination of aqueous and organic solvent or carrier. The monomers used to form the monoethylenically unsaturated polymer units and the polyacrylate ester units are added to the solution and polymerized using a template polymerization process in which the polyolefin glycol or oxide serves as a template polymer. Before initiation, the polar groups of the monomers, for instance the acid groups of acrylic acid, are attracted to the polyolefin glycol and/or polyolefin oxide through hydrogen bonding. The steric alignment of the monomers, with the polyolefin glycol and/or oxide serving as backbone, aids in the polymerization and typically increases the chain length of the polymerizing unit. During the polymerization, radical polymerizing chains may become attached to the template polymer, resulting in grafting of polyolefin glycol and/or oxide to the copolymer being formed. However, this graft polymerization need not occur. The resulting absorbent binder composition includes the polyolefin glycol and/or oxide attached to, and/or blended with, the copolymer of the monoethylenically unsaturated polymer units and the acrylate or methacrylate ester units that include the alkoxysilane functionality.

The polymerization may be initiated using a variety of methods, including without limitation thermal energy, ultraviolet light, and redox chemical reactions. A solution of the above ingredients may be added to an initiator solution at a temperature suitable for generating free radicals, for instance about 50 to about 90° C. An initiator may be prepared by dissolving an initiator in an organic or aqueous solvent. Suitable classes of initiators are organic peroxides and azo compounds, with benzoyl peroxide and azobisisobutylnitrile (ABN) as examples.

Compounds containing an O—O, S—S, or N═N bond may be used as thermal initiators. Compounds containing O—O bonds; i.e., peroxides, are commonly used as initiators for polymerization. Such commonly used peroxide initiators include: alkyl, dialkyl, diaryl and arylalkyl peroxides such as cumyl peroxide, t-butyl peroxide, di-t-butyl peroxide, dicumyl peroxide, cumyl butyl peroxide, 1,1-di-t-butyl peroxy-3,5,5-trimethylcyclohexane, 2,5-dimethyl-2,5-di(t-butylperoxy)hexane, 2,5-dimethyl-2,5-bis(t-butylperoxy)hexyne-3 and bis(a-t-butyl peroxyisopropylbenzene); acyl peroxides such as acetyl peroxides and benzoyl peroxides; hydroperoxides such as cumyl hydroperoxide, t-butyl hydroperoxide, p-methane hydroperoxide, pinane hydroperoxide and cumene hydroperoxide; peresters or peroxyesters such as t-butyl peroxypivalate, t-butyl peroctoate, t butyl perbenzoate, 2,5-dimethylhexyl-2,5-di(perbenzoate) and t-butyl di(perphthalate); alkylsulfonyl peroxides; dialkyl peroxymonocarbonates; dialkyl peroxydicarbonates; diperoxyketals; ketone peroxides such as cyclohexanone peroxide and methyl ethyl ketone peroxide. Additionally, azo compounds such as 2,2′-azobisisobutyronitrile abbreviated as AIBN, 2,2′-azobis(2,4-dimethylpentanenitrile) and 1,1′-azobis(cyclohexanecarbonitrile) may be used as the initiator.

Alternatively, redox initiation can be used for the polymerization. This method incorporates a first monomer solution that includes a reducing polymerization initiator. Suitable reducing polymerization initiators include, but are not limited to, ascorbic acid, alkali metal sulfites, alkali metal bisulfites, ammonium sulfite, ammonium bisulfite, alkali metal hydrogen sulfite, ferrous metal salts such as ferrous sulfates, sugars, aldehydes, primary and secondary alcohols, and combinations thereof. In one embodiment, the reducing polymerization initiator includes ascorbic acid.

The second monomer solution further includes an oxidizing polymerization initiator. Suitable oxidizing initiators include, but are not limited to, hydrogen peroxide, alkali metal persulfates, ammonium persulfate, alkylhydroperoxides, peresters, diacryl peroxides, silver salts, and combinations thereof. In one embodiment, the oxidizing polymerization initiator includes hydrogen peroxide.

Generally, when the first aqueous monomer solution is combined with the second aqueous monomer solution the reducing polymerization initiator reacts with the oxidizing polymerization initiator, e.g., a redox reaction, thereby initiating a polymerization reaction to form a binder composition including a monoethylenically unsaturated monomer and an ethylenically unsaturated monomer that has post-application, moisture-induced crosslinking capability.

In one embodiment, the monoethylenically unsaturated polymer unit is a cationic polymer. The cationic polymer is advantageous because it provides inherent antimicrobial properties. Suitable cationic polymers include those prepared by copolymerizing a monomer 1) selected from a) acryloyloxyethyl-trialkyl-substituted ammonium salts, b) acryloyloxypropyl-trialkyl-substituted ammonium salts, c) acrylamidoethyl-trialkyl-substituted ammonium salts, and d) acrylamidopropyl-trialkyl-substituted ammonium salts, with a monomer 2) selected from a) methacryl esters which contain an alkoxysilane group capable of moisture-induced crosslinking and b) acryl esters which contain an alkoxysilane group capable of moisture-induced crosslinking. Other monomers may also be present, for instance, an acrylic acid or acrylamide. The polymerization is conducted in the presence of a polyolefin glycol and/or polyolefin oxide as described above, suitably a polyethylene glycol. The cationic monoethylenically unsaturated monomer unit and the polyolefin glycol are present in the amounts described above.

In one embodiment, the absorbent binder composition is made by combining a first aqueous monomer solution including a reducing polymerization initiator with a second aqueous monomer solution including an oxidizing polymerization initiator, wherein the initiators react to form the absorbent binder composition. The first aqueous monomer solution further includes a monoethylenically unsaturated monomer and an ethylenically unsaturated monomer that contains an alkoxysilane functionality. The second aqueous monomer solution includes a monoethylenically unsaturated monomer. One or both solutions may include the polyolefin glycol and/or polyolefin oxide template polymer. Suitably, the absorbent binder composition is formed in about 100 minutes or less, or about 60 minutes or less, desirably in about 30 minutes or less, or about 15 minutes or less, or about 10 minutes or less.

The pH of the first and/or second aqueous monomer solution is adjusted to about 4.5 to about 8, suitably about 5.5 to about 7.0. The pH of the first aqueous solution may be adjusted prior to the addition of the ethylenically unsaturated monomer. Desirably, the pH of the first aqueous monomer solution is adjusted prior to the addition of the reducing polymerization initiator. The pH of the second aqueous solution may be adjusted prior to the addition of the oxidizing polymerization initiator. Alternatively, the pH of the combined first and second aqueous monomer solutions may be adjusted to about 4.5 to about 8, suitably about 5.5 to about 7.0.

The amounts of the polymerization ingredients added to the first and second aqueous solutions are selected so as to produce the absorbent binder composition having the composition described above. In one embodiment, a surfactant may be added to the first and/or second aqueous monomer solution to disperse the ethylenically unsaturated monomer.

The first aqueous monomer solution further includes a reducing polymerization initiator. Suitable reducing polymerization initiators include, but are not limited to, ascorbic acid, alkali metal sulfites, alkali metal bisulfites, ammonium sulfite, ammonium bisulfite, alkali metal hydrogen sulfite, ferrous metal salts such as ferrous sulfates, sugars, aldehydes, primary and secondary alcohols, and combinations thereof. In one embodiment, the reducing polymerization initiator includes ascorbic acid.

The second aqueous monomer solution further includes an oxidizing polymerization initiator. Suitable oxidizing initiators include, but are not limited to, hydrogen peroxide, alkali metal persulfates, ammonium persulfate, alkylhydroperoxides, peresters, diacryl peroxides, silver salts, and combinations thereof. In one embodiment, the oxidizing polymerization initiator includes hydrogen peroxide.

Generally, when the first aqueous monomer solution is combined with the second aqueous monomer solution the reducing polymerization initiator reacts with the oxidizing polymerization initiator, e.g. a redox reaction, thereby initiating a polymerization reaction to form the absorbent binder composition including a monoethylenically unsaturated monomer and an ethylenically unsaturated monomer that has post-application, moisture-induced crosslinking capability.

When placed on a substrate and crosslinked, the absorbent binder composition forms an absorbent layer on the substrate or is impregnated into the substrate. As described above, the absorbent binder composition forms a superabsorbent layer on the substrate or a superabsorbent within the substrate. As with the substrate, it is desirable that the absorbent layer has a high level of light transmittance. The level of light transmittance of the absorbent material is not critical to the present invention, so long as the absorbent composite containing the substrate and the absorbent layer has a light transmittance of at least 45%, as described above. At the same time, it is desirable that the absorbent material have a light transmittance as high as possible. Ways to control the light transmittance of the absorbent layer include reducing or eliminating the amounts of coloring agents, pigments, fillers and other materials which may cause a reduction of light transmittance from the raw materials used to make the absorbent binder composition. In one embodiment of the present invention, the absorbent binder composition contains less than about 2% fillers, pigments or coloring agents which can reduce the light transmittance of the resulting absorbent layer. Desirably, the absorbent binder composition contains less than about 0.5% by weight of fillers, pigments or coloring agents which can reduce the light transmittance of the resulting absorbent material of the composite. Most desirably, the absorbent binder composition is substantially free of coloring agents, pigments, filler and other similar materials which may reduce the light transmittance of the resulting absorbent layer.

Other ways to improve or keep the light transmittance of the absorbent material and hence the absorbent composite in the desired range described above is to keep the basis weight of the absorbent material in the composite or thickness of any resulting absorbent layer to a minimum, while providing sufficient absorbency to the absorbent articles. While keeping the thickness and basis weight to a minimum, care should be taken so the absorbent composite has sufficient absorbing capacity. In the present invention, when the absorbent material should have a basis weight of about 2 gsm to about 200 gsm, on a solids basis and/or the layer should have a thickness less than 1.0 mm (broad thickness). Desirably, the absorbent material should have a basis weight between about 5 gsm and about 100 gsm, on a solids basis or a thickness of less than about 0.5 mm. Most desirably, the absorbent material should have a basis weight between about 10 gsm and about 75 gsm, on a solids basis, or a thickness of less than about 0.3 mm, typically in the range of about 0.01 mm to about 0.3 mm As the thickness or basis weight of the absorbent material increases, the light transmittance of the absorbent composite may be reduced. On the other hand, if the basis weight or thickness of the absorbent material is reduced, the capacity of the absorbent composite may be reduced. It has been discovered that if the absorbent binder is applied in these ranges, the absorbent layer formed from the absorbent binder will have sufficient absorbency for most intended uses of the absorbent composite, while still providing a degree of translucence to the composite so that the absorbent composite is translucent and will blend in with the use environment. In addition, the amount of the absorbent binder may be changed to meet a desired or needed absorbency for the absorbent composite.

The substrate 11 may be a liquid permeable material or a liquid impermeable material. When the absorbent material 12′ is formed on a liquid permeable substrate 11, the absorbent composite 10 may need an additional liquid impermeable layer for the absorbent composite to be able to fully contain and hold an insulting fluid. As a result, the absorbent composite of the present invention may contain at least one additional layer. The additional layer may be a liquid permeable material or a liquid impermeable material. Suitable additional layers include both liquid permeable materials or liquid impermeable materials. The additional layer may be a film, a nonwoven web knitted fabric or a woven fabric, or a laminate of one or more of these materials. The only requirements for the additional layer is that the additional layer has a light transmittance of at least 45%. In addition, the additional layer should have sufficient flexibility so the absorbent can be used in flexible absorbent personal care articles. Particular examples of substrates include, polyolefin films, spunbond nonwoven webs and laminates of polyolefin films and spunbond nonwoven webs, bonded carded webs, bonded airlaid webs, coform, and woven fabrics such as cotton and wool cloths. There may be more than one additional layer present in the absorbent composite. Generally, it is desired that the one or more additional layers each have a light transmittance of at least 60%. As with the substrate layer, in an embodiment of the present invention, the additional layer contains less than about 2% fillers, pigments or coloring agents which can reduce the light transmittance of the additional layer. Desirably, the additional layer contains less than about 1% by weight of fillers, pigments or coloring agents which can reduce the light transmittance of the additional layer. Most desirably, the additional layer is substantially free of coloring agents, pigments, fillers and other similar materials which may reduce the light transmittance of the additional layer.

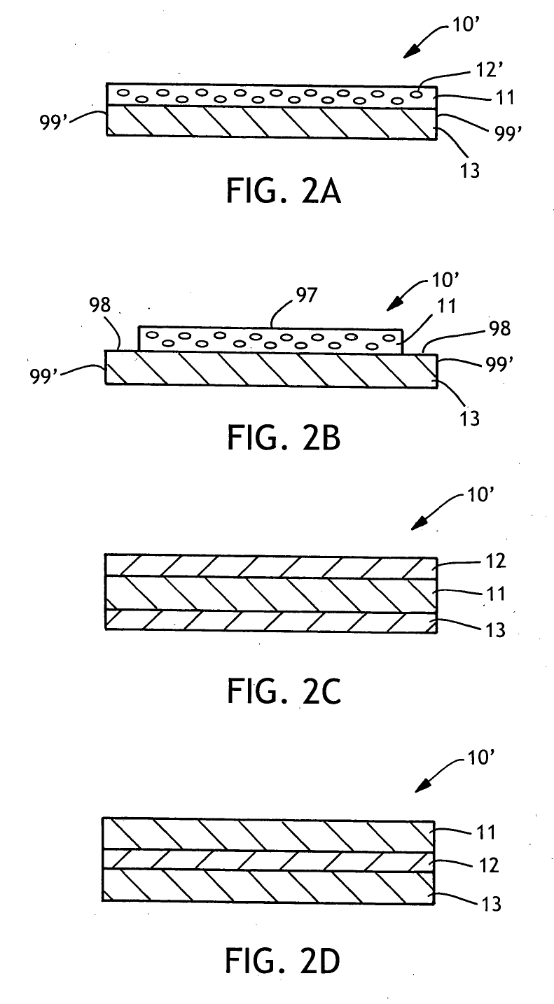

To obtain a better understanding of the absorbent composite with additional layers, attention is directed to FIGS. 2A and 2B. FIG. 2A shows an absorbent composite 10′ having a substrate layer 11 impregnated with the absorbent material 12′ and an additional layer 13. The additional layer 13 is adjacent the substrate 11 with the absorbent material 12′ impregnated therein. As is shown in FIG. 2A, the substrate 11 with the absorbent material 12 impregnated therein is coextensive with the side edges 99′ of the additional layer 13. In an alternative embodiment, shown in FIG. 2B, the substrate 11 having the absorbent material 12′, applied therein positioned on the additional layer 13 such that the substrate and absorbent material therein is not coextensive with the edges 99 of the additional layer. As stated above in regard to FIG. 1C, it is noted that the absorbent material 12′ appears to be shown in FIGS. 2A and 2B as a discrete phase or discrete particles. However the intent is to show that the absorbent material 12′ is impregnated within the substrate 11. That is, the absorbent material could be a continuous phase within the substrate 11.

In addition, the substrate 11 may have the absorbent material 12′ applied as an additional layer on the substrate 12 as is shown in FIG. 2C. The additional layer 13 may be bonded to the substrate layer 11 using a known technique, such as, adhesive bonding, pattern bonding using heat and pressure, ultrasonic bonding, stitching and other similar joining techniques. The layers of the absorbent composite may be held together using suitable bonding techniques, including those described above. In another alternative embodiment, the absorbent material 12 from the absorbent binder composition may hold the additional layer 13 to the substrate 11 as is shown in FIG. 2D. When a three layer structure absorbent composite is desired or prepared, the absorbent binder composition may be applied to one of layers 11 or 13 or both layers 11 and 13. The layers are brought together so that the absorbent binder composition contacts each layer of the substrate 11 and additional layer 13 of the absorbent composite. As a result, the absorbent binder and the resulting absorbent layer 12 are directly joined to the adjacent substrate 11 and additional layer, without an additional adhesive. This is accomplished by applying an absorbent binder composition to facing surfaces of one or both layers 11 and 13, bringing the layers 11 and 13 together so that the absorbent binder composition contacts both layers, and crosslinking the absorbent binder composition to form the absorbent layer 12. Crosslinking can be moisture-induced by hydrolysis and condensation of alkoxysilanes. For example, crosslinking of the absorbent binder composition can be induced by concentrating the binder composition through the removal of the water to promote condensation of silanols generated by hydrolysis of alkoxysilanes.

The absorbent binder layer may be formed on the substrate or support layer as a continuous layer having uniform thickness, or as a discontinuous or nonuniform layer which provides flow channels, liquid retention dams, or other desired attributes. However, because the absorbent layer 12 is intended as a sole or primary absorbent layer in the simplified absorbent article, the flexible absorbent binder should be present in sufficient thickness and quantity, and over a sufficient area to provide substantially all of the liquid absorption capacity that is required by the end use application. Alternatively, superabsorbent particles can be incorporated into the absorbent binder and hence the absorbent layer 12 formed from the absorbent binder, to provide a portion of the liquid absorption capacity required by the end use application.

Because the flexible absorbent binder is in contact with layers 11 and 13 as it is being formed, the resulting absorbent layer 12 adheres to the substrate layer and the additional layer 13 in addition to serving as an absorbent (fluid storage) layer. Thus, the absorbent composite 10′ of the invention provides three layers bound together in sequence, namely a fluid receiving layer or backing layer, a flexible absorbent binder layer, and a support layer, without intervening adhesive layers.

In another embodiment, the absorbent binder composition may be prepared using a continuous process wherein the polymerization and/or neutralization reaction is carried out in a suitable reactor that conveys the resulting binder composition, upon completion of the polymerization reaction, directly to an apparatus for applying the absorbent binder composition onto the substrate layer 11 and/or the additional layer 13. Such a continuous process may be desirable where conditions, such as high heat, may cause premature crosslinking of the binder composition that would hinder application of the absorbent binder composition onto the substrate.

One advantage of the absorbent binder composition is that it provides a water-soluble ionic polymer capable of sufficient spontaneous crosslinking within about 10 minutes, at a temperature not more than about 150° C., to provide the flexible absorbent binder layer with an absorbent capacity of at least one gram of fluid per gram of flexible absorbent binder layer, suitably at least three grams of fluid per gram of flexible absorbent binder layer, using the centrifuge retention capacity test. As used herein, the Centrifuge Retention Capacity (CRC) is a measure of the absorbent capacity of the superabsorbent material retained after being subjected to centrifugation under controlled conditions. The CRC can be measured by placing a sample of the material to be tested into a water-permeable bag which will contain the sample while allowing the test solution (0.9 percent NaCl solution) to be freely absorbed by the sample. A heat-sealable tea bag material (available from Dexter Nonwovens of Windsor Locks, Conn., U.S.A., as item #1234T) works well for most applications. The bag is formed by folding a 12.7 cm by 7.6 cm sample of the bag material in half and heat sealing two of the open edges to form a 6.3 cm by 7.6 cm rectangular pouch. The heat seals should be about 6 mm inside the edge of the material. After the sample is placed in the pouch, the remaining open edge of the pouch is also heat-sealed. Empty bags are also made to be tested with the sample bags as controls. A sample size is chosen such that the teabag does not restrict the swelling of the material, generally with dimensions smaller than the sealed bag area (about 5.1 cm by 6.3 cm). Three sample bags are tested for each material. The sealed bags are submerged in a pan of 0.9% NaCl solution. After wetting, the samples remain in the solution for 60 minutes, at which time they are removed from the solution and temporarily laid on a non-absorbent flat surface. The wet bags are then placed into the basket of a suitable centrifuge capable of subjecting the samples to a g-force of 350. (A suitable centrifuge is a Heraeus LABOFUGE 400, Heraeus Instruments, part number 75008157, available from Heraeus Infosystems GmbH, Hanau, Germany). The bags are centrifuged at 1600 rpm for 3 minutes (target g-force of 350). The bags are removed and weighed. The amount of fluid absorbed and retained by the material, taking into account the fluid retained by the bag material alone, is the Centrifugal Retention Capacity of the material, expressed as grams of fluid per gram of material.

The term “spontaneous” crosslinking refers to crosslinking which occurs without radiation, catalysis, or any other inducement other than the specified temperature of not more than about 150° C., suitably not more than about 120° C. Eliminating the need for radiative crosslinking provides a significant processing advantage. The crosslinking at temperatures not more than about 120° C., suitably not more than about 100° C., permits the absorbent binder composition to be applied to one or more substrate layers, and then crosslinked without degrading or damaging the substrate. Significant crosslinking occurs within about 10 minutes, suitably within about 8 minutes, particularly within about 6 minutes provides an efficient, commercially feasible, cost-effective crosslinking process. The crosslinking may then continue until flexible absorbent polymer having the desired absorbent capacity is obtained. The ionic polymer may bear a positive charge, a negative charge, or a combination of both, and should have an ionic unit content of about 15 mole percent or greater. The ionic polymer may include a variety of monomer units described above, and suitably contains a carboxyl group-containing unit or a quaternary ammonium-containing unit.

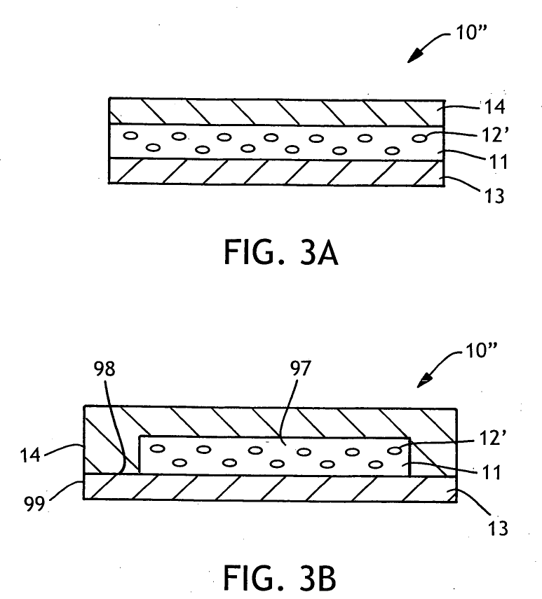

In another embodiment of the present invention, the absorbent composite may have a second additional layer 14. In this regard, attention is directed to FIG. 3A. The second additional layer 14 may be any of the same materials described above for the first additional layer 13. When the second additional layer is present, generally the substrate 11 and the absorbent material 12′ applied thereto are positioned between the first additional layer 13 and the second additional layer 14. Generally when two additional layers are present, one of the additional layers is a liquid impenmeable material and the other additional layer is a liquid permeable material. As with the first additional layer, it is generally desirable that the second additional layer have a light transmittance of at least 60%. As with the substrate layer and the first additional layer, in an embodiment of the present invention, the second additional layer contains less than about 2% fillers, pigments or coloring agents which can reduce the light transmittance of the second additional layer. Desirably, the second additional layer contains less than about 1% by weight of fillers, pigments or coloring agents which can reduce the light transmittance of the second additional layer. Most desirably, the second additional layer is substantially free of coloring agents, pigments, fillers and other similar materials which may reduce the light transmittance of the second additional layer. More desirable, the first and second additional layers each have a light transmittance of at least 80% and the overall absorbent composite has a light transmittance between about 65 and 79%.

In another embodiment of the present invention, the additional layer forms a backing layer of the composite. The backing layer serves to prevent any fluids absorbed by the substrate 11 and the absorbent material 12′ applied thereon from passing through the absorbent composite 10″. Generally, the backing layer is fluid impermeable. In addition, in a further embodiment of the present invention, the second additional layer serves as a liner layer of the composite. The liner layer protects the substrate and the absorbent material applied thereon during use of the absorbent composite. In addition, the liner may serve to protect the user of the absorbent composite 10″ from having direct contact with the superabsorbent material that may be present in the absorbent material 12′.

In another aspect of the present invention, the absorbent composite may have two distinct areas of the composite which have different translucence, meaning different light transmittance. To obtain a better understanding of this aspect of the present invention, attention is again directed to FIG. 2B, which shows an absorbent composite having a central region 97 and a perimeter region 98. The central region includes both the additional layer 13, which is typically a backing layer, in which the substrate 11 and the absorbent material 12′ applied thereon is adjacent the additional layer. The perimeter region 98 only included the additional layer 13 or backing layer. In this embodiment of the present invention, the perimeter region of the absorbent composite desirably has a light transmittance of at least 60% and the central region desirable has a light transmittance of at least 45%. More desirably, the perimeter region of the absorbent composite has a light transmittance of at least 80% and the central region desirably has a light transmittance between about 65% and 79%. By having a difference in light transmission, a user of the absorbent composite can see the area of the composite which has the absorbency. Similarly, in an additional embodiment of the present invention shown in FIG. 3B, when the second additional layer is present, the second additional layer 14 or typically the liner layer, and the first additional layer 13 or the backing layer are present in the perimeter region 98 and the substrate 11 with the absorbent material 12′ applied thereto along with the first and second additional layer 13, 14 are present in the central region. The central region 97 and perimeter region 98 of the absorbent composite have light transmission properties described above. As stated above in regard to FIG. 1C, it is noted that the absorbent material 12′ appears to be shown in FIGS. 3A and 3B as a discrete phase or discrete particles. However the intent is to show that the absorbent material 12′ is impregnated within the substrate 11. That is, the absorbent material could be a continuous phase within the substrate 11.

The absorbent composites of the present invention are relatively thin and can have thickness as low as about 0.05 mm and thickness as high as 5 mm or more at a pressure of 1.35 kPa. Generally, it is desirable that the absorbent composites be as thin as possible while providing sufficient absorbency. Typically the absorbent composites of the present invention have a thickness in the range of about 0.1 mm to about 2.0 mm and more typically about 0.2 to about 1.2 mm. In addition, the absorbent composites of the present invention have an absorbency up to about 10 g/g of the absorbent composite. Typically, the absorbent composite of the present invention will absorb between about 0.8 g/g to about 5 g/g of the absorbent composite.

The translucent absorbent composite of the present invention also has the property of becoming soft and pliable under close-to-the-body conditions. The absorbent binder composition is a very hydrophilic material with the ability to absorb water vapor. This property provides a benefit for thin absorbent articles because the relative stiffness of the article, when removed from the wrapper, allows the user to place the article in the undergarment with ease. However, when placed close to the body, the article becomes softer and more body conforming as a result of uptake of water vapor into the absorbent composite. This makes the absorbent composites of the present invention useable in absorbent articles, especially those absorbent articles used in sanitary napkins, pantiliners, diapers and the like.

The translucent absorbent composite of the present invention can be used as an absorbent component or absorbent layer in a wide variety of absorbent articles including, but not limited to, sanitary napkins, pantiliners, bandages, bed liners, furniture liners as well as other absorbent articles that need both absorbency and transparency or translucence. Typically, absorbent articles have an absorbent layer, and a backing layer, which helps retain any absorbed fluids in the absorbent article. Most absorbent articles have a backing layer which is a liquid impermeable layer. The backing layer generally faces away from the fluid source, meaning that the absorbent layer is positioned between the fluid source and the backing layer. In some applications, such as a bandage, the backing layer may be apertured material, such as an apertured film, or material which is otherwise gas permeable, such as gas permeable films. In absorbent personal care articles such as pantiliners, the backing layer which is a liquid impermeable layer is usually the garment facing layer. The backing layer is often referred to as the backsheet, baffle or outercover. Additional layers, such as a liner, also commonly referred to as a bodyside liner may also be present in the absorbent article of the present invention.

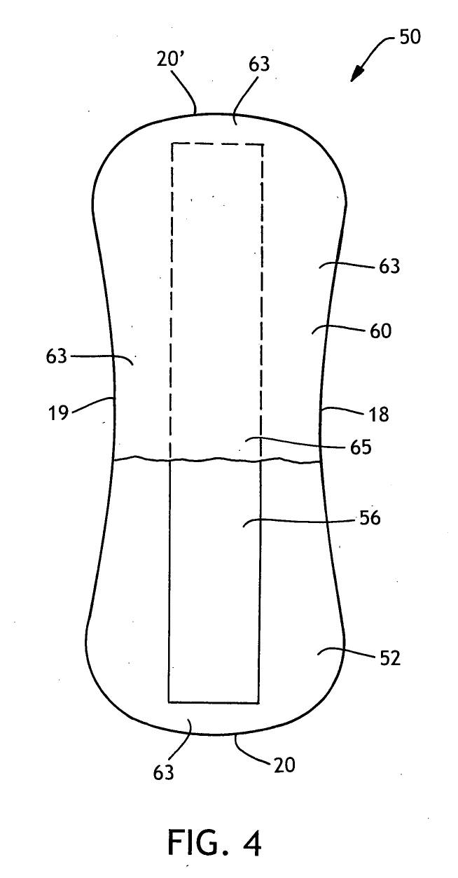

In the present invention, the absorbent article is translucent, meaning that the absorbent article has a minimum light transmittance of about 45%. The absorbent layer of the absorbent article is prepared from an absorbent composite described above. In particular the absorbent layer is a substrate having an absorbent binder composition, describe above, applied to the substrate. In an absorbent article prepared from the absorbent composite of the present invention, the absorbent layer of the absorbent article may contain the translucent absorbent composite as the main absorbent structure of the absorbent article. Layers of the absorbent composite of the present invention may also function as a layer of the absorbent article. For example, if the substrate layer 11 is a liquid impermeable material, the substrate layer of the absorbent composite could also function as the liquid impermeable layer of the absorbent article, for example, the backing layer of the absorbent article. To obtain a better understanding of an absorbent article of the present invention, attention is directed to FIG. 4. This figure illustrates an absorbent article 50 formed using an absorbent composite of the present invention. The absorbent article 50, as illustrated, includes three layers. These layers include, a backing layer 52, also commonly referred to as a backsheet, baffle or outercover; an absorbent layer 56, which is formed from an absorbent composite 54 of the present invention, the absorbent composite is a substrate with the flexible absorbent binder composition applied thereto; and an optional bodyside liner layer 60, also commonly called a liquid intake layer. In the present invention, it is desirable that the bodyside liner layer 60 is present in the absorbent article. Typically, the backing layer 52 of the absorbent article is a liquid impermeable layer made from a liquid impermeable material and the bodyside liner layer 60 is a liquid permeable layer and is prepared from a liquid permeable material.

In the present invention, the absorbent article contains the absorbent composite described above. In addition, the absorbent article of the present invention has a minimum light transmittance of at least 45% as measured by the BYK-Gardner Haze Guard Plus in accordance with ASTM-D1003-00. Desirably, the absorbent article has a minimum light transmittance of at least 50% and generally has a minimum light transmittance in the range of about 60% to about 79%.

The absorbent article 50 may include only two layers 52 and 56, or desirably the three layers 52, 56 and 60. Optionally, other layers may be included in the absorbent article on or both sides of the absorbent layer. If additional layers are present in the absorbent article, the additional layers should not adversely effect the light transmission through the absorbent article. That is, the minimum light transmittance should be at least 45%. In any case, the absorbent article 50 of the present invention will have a simplified construction compared to conventional absorbent articles because a) the absorbent binder containing absorbent composite 56 (with or without superabsorbent particles) provides essentially all of the required absorbent capacity, and b) the absorbent binder layer 56 may bind to the adjacent layers 52 and 60 without intervening adhesive layers.

Referring again to FIG. 4, the support layer 52 may be a liquid-impermeable outer cover material. Suitable outer cover materials include, without limitation, polyolefin films (e.g., films of polypropylene and polyethylene homopolymers and copolymers), breathable polyolefin films (e.g., stretch-thinned films formed from one or more polyolefins), and laminates of a breathable polyolefin film and a polyolefin nonwoven web (e.g., a spunbond web). Alternatively, the absorbent article 50 may be designed to include one or more functional layers, such as a dampness-inhibiting “spacer” structure, between the outer cover and the flexible absorbent polymer layer 56. In such instances, the support layer 52 may be any layer that is positioned directly below the flexible absorbent polymer layer 52 in the absorbent article 50. Depending on the application, the support layer 52 may be a nonwoven web, woven web, knitted fabric layer, cellulose layer, plastic film, plastic foam, staple fiber layer, elastomeric net composite, stranded composite or another suitable material.

The bodyside liner 60 may be an apertured film, an open nonwoven layer such as a spunbond layer, bonded-carded web or staple fiber web, an open-celled (e.g., reticulated) foam, a cellulose web, or any suitable open structure capable of receiving and/or distributing liquid. The fluid-receiving layer 60 may be homogeneous in the thickness direction or have a gradient structure. The desired composition of fluid receiving layer 60 may depend on whether the fluid-receiving layer 60 is used as a bodyside liner, or whether it is an interior fluid-receiving layer (e.g., a surge/transfer or compensation layer) used in addition to one or more other fluid receiving layers.

Other features may be present on the absorbent personal care article. The absorbent product 50 also has a first side 18 and a second side 19. The first and second sides 18, 19, respectively, are the longitudinal sides of the elongated absorbent product. The sides can be contoured, for example, in a concave shape as shown in FIG. 3, or they can be linear. The sides can further include flaps (not shown) that extend laterally outward. Flaps are known in the art and are shown in, for example, U.S. Pat. No. 6,387,084 issued to VanGompel et al. or U.S. Pat. No. 4,589,876, issued to Van Tillburg, which are hereby incorporated by reference for its discussion of the flaps and flap attachment means and in its entirety. In one embodiment (not shown), one or more elastic elements are disposed along the sides to form a gasket with the body of the user. Elastic sides are known in the art, as is shown in U.S. Pat. No. 6,315,765 issued to Datta et al., which is hereby incorporated by reference for its discussion of the elastic sides and in its entirety. In one embodiment, the elastic elements are disposed between the liner and the outer cover. If these additional flaps are present, it is desirable that the flaps have a light transmittance of at least 60%, and desirably at least 80%.

In another embodiment of the present invention, the absorbent article 50 has a perimeter region 63 and a central region 65. The backing layer 52 and the bodyside liner 60, when present, are each present in both the perimeter region 63 and the central region 65. The absorbent layer 56 is only present in the central region 65 of the absorbent article 50. The perimeter region 63 may completely surround the central region 65, as is shown in FIG. 4, or the perimeter region 63 may be either side of the central region, such that central region extends to the ends of the absorbent article 20, 20′. In this embodiment of the present invention, the perimeter region may have a light transmittance of at least 60% and the central region may have a light transmittance of at least 45%. In another aspect of this embodiment of the present invention, the perimeter region 63 of the absorbent article 50 has a light transmittance of at least 80% and the central region 65 of the absorbent article 50 has a light transmittance of between about 65% and about 79%. It is desirable that the light transmission in the absorbent area be different from the light transmission in the regions surrounding the absorbent, since the difference in light transmission allows the user to see the absorbent, giving the user confidence that the liner will function as intended.

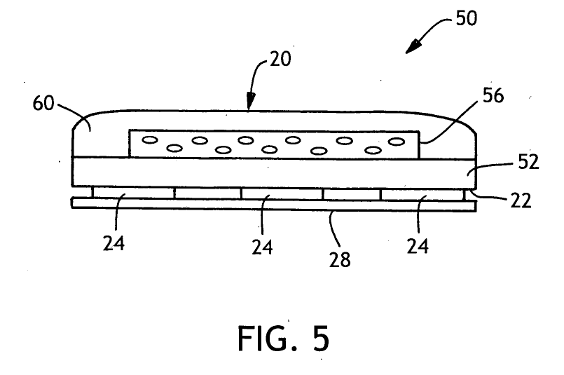

Referring to FIG. 5, shown is a cross-section of the absorbent article 50. The absorbent article has a first body-side surface 20 and a second garment side surface 22. Applied to at least a portion of the second garment side surface 22 is a garment attachment adhesive 24. In various embodiments, the garment attachment adhesive 24 is configured as a single band of adhesive or as two or more spaced apart strips. Alternatively, the garment attachment adhesive 24 includes a swirl pattern of adhesive which encompasses a major portion of the second garment surface 22 of the absorbent article 50. As stated above in regard to FIG. 1C, it is noted that the absorbent material appears to be shown in FIG. 5 as a discrete phase or discrete particles. However the intent is to show that the absorbent material is impregnated within the substrate 11. That is, the absorbent material could be a continuous phase within the substrate 11.

A release strip 28, also known as a releasable peel strip, or simply a peel strip, may be removably secured to the garment attachment adhesive 24 and serves to prevent premature contamination of the adhesive 24 before the absorbent article 50 is secured to, for example, the crotch portion of an undergarment. In various embodiments, the garment attachment adhesive is designed to be secured to the inner crotch portion of an undergarment so as to keep the absorbent product in register with the body of the user. The release strip 28 may extend beyond one or both of the ends 20, 20′ of the backing layer. In another embodiment, the release strip may have a tab or other device to allow the user to see and grab the release strip so that the absorbent article 50 can be applied to an undergarment of the used environment after the adhesive 24 is exposed.

In an additional embodiment of the present invention, the release strip 28 and the garment adhesive 24 also have a light transmission of at least 60%. In this aspect, the strip may be prepared from a clear polymer film, which may have a pattern or words printed thereon so that the peel strip can be seen and removed by the user. Desirably, the release strip 28 and the garment adhesive 24 each have a light transmission of at least 80%. With the peel strip, garment adhesive, backing layer, absorbent layer and optionally the bodyside liner, the absorbent article should have a minimum light transmission of at least 45%, desirably at least 60% and most desirably in the range of about 60% to about 79%.

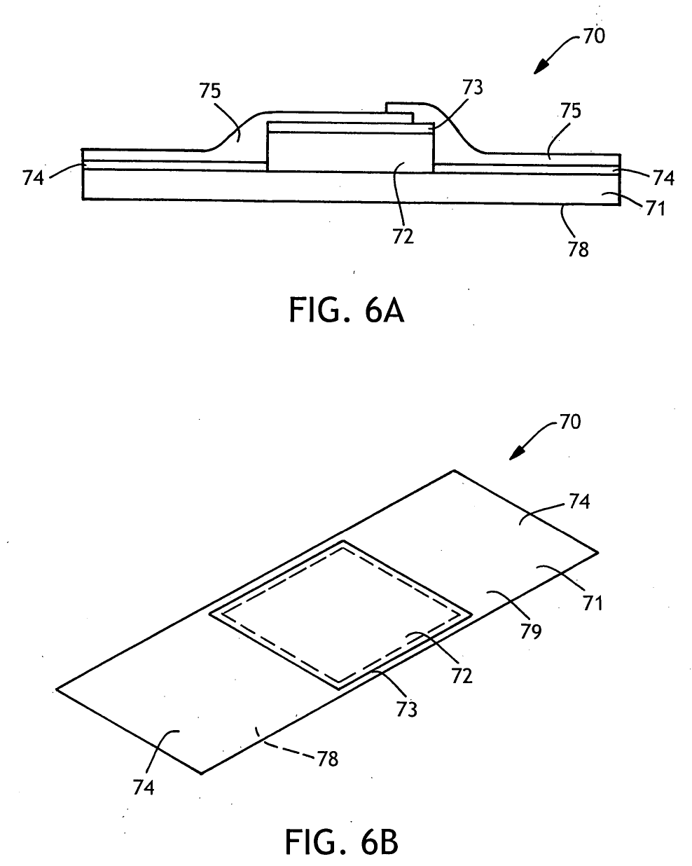

In addition to absorbent personal care articles described above, the absorbent articles of the present invention may be used as an absorbent bandage. Attention is directed to FIGS. 6A and 6B, which show a possible configuration for a bandage of the present invention. FIG. 6A shows a cross-section view of the absorbent bandage with optional layers describe below. FIG. 6B shows a perspective view of the bandage of the present invention with some of the optional or removable layers not being shown. The absorbent bandage 70 has strip 71 of material having a body-facing side 79 and a second side 78 which is opposite the body-facing side. The strip is essentially a backing layer and is desirably prepared from the same materials described above for the backing layer. In addition, the strip may be apertured material, such as an apertured film, or material which is otherwise gas permeable, such as gas permeable films. The strip 71 supports an absorbent layer 72 which is attached to the body facing side 79 of the strip. In addition, an optional absorbent protective layer 73, may be applied to the absorbent layer and can be coextensive with the strip 71. The absorbent layer contains the absorbent composite of the present invention. In the present invention, the strip is desirably translucent, having a light transmission of at least 60%. The absorbent bandage of the present invention has a minimum light transmission in the area of the absorbent layer 72 of at least 45%.

The absorbent bandage 70 of the present invention may also have a pressure sensitive adhesive 74 applied to the body-facing side 79 of the strip 71. Any pressure sensitive adhesive may be used, provided that the pressure sensitive adhesive does not irritate the skin of the user. Desirably, the pressure sensitive adhesive is a convention pressure sensitive adhesive which is currently used on similar bandages. This pressure sensitive adhesive is preferably not placed on the absorbent layer 72 or on the absorbent protective layer 73 in the area of the absorbent layer 72. If the absorbent protective layer is coextensive with the strip 71, then the adhesive may be applied to areas of the absorbent protective layer 73 where the absorbent layer is not located. By having the pressure sensitive adhesive on the strip 71, the bandage is allowed to be secured to the skin of a patient in need of the bandage. To protect the pressure sensitive adhesive and the absorbent, a release strip 75 is placed on the body facing side 79 of the bandage. The release liner may be similar to the release liner described above and may be placed on the body facing side of the bandage in a single piece (not shown) or in multiple pieces, as is shown in FIG. 6A.

In an alternative embodiment of the present invention, the absorbent layer of the bandage may be placed between a folded strip. If this method is used to form the bandage, the strip needs to be fluid permeable

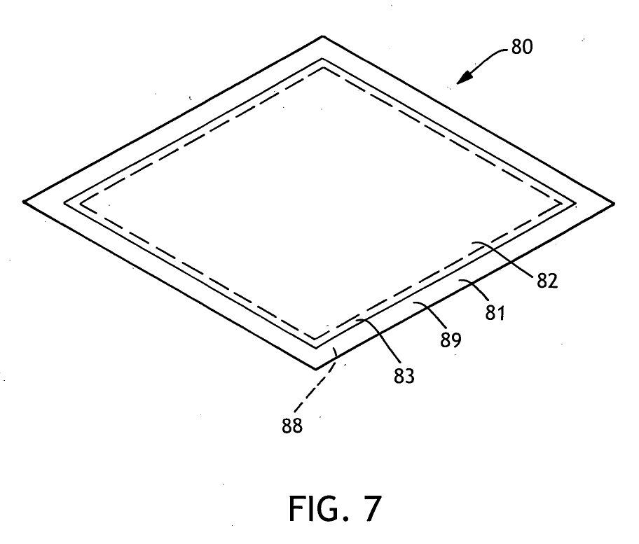

Absorbent furniture and/or bed pads are also included within the present invention. As is shown in FIG. 7, a furniture or bed pad 80, hereinafter referred to a “pad” is shown in perspective. The pad 80 has liquid impermeable backing layer 81 having a furniture-facing side or surface 88 and upward facing side or surface 89 which is opposite the furniture-facing side or surface 88. The liquid impermeable backing layer 81 supports an absorbent layer 82 which is attached to the upward facing side 89 of the liquid impermeable backing layer. In addition, an optional absorbent protective layer 83, may be applied to the absorbent layer. The absorbent layer contains the absorbent composite of the present invention. The substrate layer of the absorbent composite can be the liquid impermeable layer 81 or the absorbent protective layer 83 of the pad. In the alternative, when the absorbent composite has three layers, the three layers of the absorbent composite can include the liquid impermeable layer 81, the absorbent layer 82 and the absorbent protective layer 83. In the present invention, the liquid impermeable layer is desirably translucent, having a light transmission of at least 60%. The absorbent pad of the present invention has a light transmission of at least 45%, as measured in the area of the pad 80 having the absorbent layer 82. Desirably, the absorbent pad 80 has a minimum light transmission of at least 60% in the area of the absorbent layer 82 and more desirably in the range of about 65% to about 79%. As a result, the absorbent pad will blend in with the material of the furniture it is used on, providing the user with a discreet means to use the furniture pad without having other easily recognize that the user is in need of the absorbent furniture pad.

To hold the pad in place, the furniture-facing side 88 of the pad may contain a pressure sensitive adhesive, a high friction coating or other suitable material which will aid in keeping the pad in place during use. The pad of the present invention can be used in a wide variety of ways including on chairs, sofas, beds, car seats and the like to absorb any fluid which may come into contact with the pad.

The absorbent articles of the present invention may be prepared by placing the absorbent composite onto a backing layer and adding the optional liner layers. In the alternative, the absorbent articles may be cut from an absorbent composite sheet having the one or more additional layers described above.

In another embodiment of the present invention, provided is an absorbent article comprising a body contacting surface, a surface opposed the body contacting surface, an absorbent core position between the body contacting surface and the surface opposed the body contacting surface, longitudinal edges extending along an edge of absorbent core and flaps. The flaps extend from the longitudinal edges of the absorbent article and the flaps contain an absorbent material, which is capable of absorbing fluids. The flaps have a have a light transmittance of at least 45%. Desirably, the light transmittance of the flaps in at least 60%. The absorbent of the flaps is formed from the absorbent binder composition described above. A portion or the entire flap may contain the absorbent binder. In an alternative configuration, the flaps of the absorbent articles may contain the absorbent composite of the present invention. In the absorbent article of this embodiment of the present invention with flaps, the absorbent layer may be an absorbent layer conventionally used in the art or may be the absorbent layer described above. In addition, the body contacting surface may be the bodyside liner described above, and the surface opposed the body-contacting surface may be the backsheet described above. Desirably, the flaps are prepared from a laminate of the body contacting surface and the surface opposed the body contacting surface.

It has been discovered thatthe absorbent composite of the present invention tends to become more flexible during use. As a result, the flexible articles containing the composite have a stiffness that convey to the user that the article has the ability to absorb and retain fluids, but at the same time become less stiff and more comfortable as the article is being used or worn.

### EXAMPLES

### Example 1

Two monomer solutions were prepared separately. Solution No. 1 was prepared as follows: to 237 grams (3.289 moles) of acrylic acid was added to 31.5 grams polyethylene glycol (mol. wt.=200) and 52.6 grams of sodium hydroxide in 350 grams of water (40% neutralization) and 1.5 grams of ascorbic acid. This solution was cooled in an ice bath.

Solution No. 2 was prepared as follows: 31.5 grams polyethylene glycol (mol. wt.=200) was diluted with 200 g water, then, with rapid stirring was added 5 ml of 3-(trimethoxysilyl)propyl methacrylate(2.7×10⁻² moles) to produce a hazy solution. To this solution was added 3.15 g of 30% aqueous hydrogen peroxide.

A third solution was prepared by dissolving 39.5 grams (0.987 moles) sodium hydroxide in 300 grams of water.

Solution No. 2 was added to Solution No. 1 in an ice bath while stirring with a magnetic stir bar. A thermocouple was used to monitor the temperature and observe the reaction exotherm. The polymerization reaction began after about 5 minutes of mixing. Once the exotherm reaction was detected, water was added gradually to keep the solution viscosity suitable for stirring. A total of 450 gram of water was added over 20 minutes. A maximum polymerization temperature of 85° C. was observed about 8 minutes after mixing of the two monomer solutions. After about 20 minutes solution 3 was added with stirring to bring neutralization to 70%, followed by additional water to reduce the polymer concentration to about 20%.

A 21 gsm spunbond containing 1.8 denier polypropylene spunbond fibers containing about 1% TiO₂ and a wire weave bond pattern which has been necked down 25%, and treated with 0.34% surfactant mixture containing Ahcovel (available from Hodgson Chemicals, Inc.) and Glucopon (available from Henkel Corporation) mixture. The spunbond was immersed in the binder solution to thoroughly saturate the fabric. Excess fluid was squeezed out, and the saturated spunbond was dried for 4 minutes at 105 degrees Celsius in a Mathis through-air-dryer oven. After drying, the coated fabric had about a 35 gsm dry add-on of the dried absorbent binder composition.

The absorbent composite was cut from the sample having a length of 12.5 cm long and 2.8 cm wide and to be used in an absorbent layer of a pantiliner. The backing sheet of the pantiliner was formed from clear film available from Pliant Corp. The bodyside liner was a 18.5 gsm polypropylene spunbond with no TiO₂ present in the polypropylene. The absorbent composite is placed between the film and the bodyside liner and the liner, film and composite are joined together using a clear adhesive. The film and bodyside liner are cut to a dog bone shape, similar to the shape shown in FIG. 4. The article has a length of 150 mm and a width at its narrowest region of 48 mm and a width at its widest region of 55 mm. The resulting absorbent article has a central region containing the absorbent and a perimeter region which surrounds the central region. The light transmission of this central region of the absorbent article was test in accordance with ASTM D-1003, it was determined that the absorbent article had an average light transmission of 68% (std. dev. 1.0), with an average haze value of 95% (std. dev. 3.3) and a clarity of 8% (std. dev. 0.2). In the perimeter region of the absorbent article, the average light transmission of 85% (std. dev. 1.6), with an average haze value of 87.5% (std. dev. 2.0) and a clarity of 15% (std. dev. 0.7).

### Example 2

Another absorbent article was prepared but instead of placing a single absorbent composite between the backing layer and the bodyside liner, two of the absorbent composites were placed on top of each other between the backing layer and the bodyside liner. As a result a two-layer absorbent composite is used as the absorbent layer. The light transmission of this central region of the absorbent article was tested in accordance with ASTM D-1603, and it was determined that the absorbent article had an average light transmission of 51% (std. dev. 0.5), with an average haze value of 97.8% (std. dev. 1.1) and a clarity of 3.2% (std. dev. 0.5). In the perimeter region of the absorbent article, the average light transmission was 85% (std. dev. 1.6), with an average haze value of 87.5% (std. dev. 2.0) and a clarity of 15% (std. dev. 0.7).

### Example 3

The absorbent article of Example 1 was further provided with a transparent film peel strip having a thickness of about 1 mil, available form Tekkote. The peel strip had an average light transmission of 93.7% (std. dev. 0.1), with an average haze value of 51.9% (std. dev. 0.9) and a clarity of 38.8% (std. dev. 0.6). A garment attachment adhesive available from National Starch and Chemical Company under NS-5602 was applied in 7 lines of adhesive at a basis weight of about 30 gsm. The light transmission test was rerun including the peel strip. It was determined that the absorbent article had an average light transmission of 62.4% (std. dev. 1.7), with an average haze value of 97.8% (std. dev. 1.1) and a clarity of 3.2% (std. dev. 0.5). In the perimeter region of the absorbent article, the average light transmission was 75.4 % (std. dev. 2.7)

### Example 4

Various binder compositions were placed on a 15 gsm spunbond to form a composite of the present invention. The compositions and substrates are shown in the TABLE below. The reduction in the stiffness is demonstrated by testing the stiffness of the absorbent composite, utilizing the Plate Stiffness described below. As shown in the TABLE below, there is a substantial reduction in stiffness when the absorbent article is exposed to close-to-the-body conditions of 80% relative humidity compared to the “dried” condition that approximates the condition of the absorbent composite as it is assembled into the absorbent articles and packaged.

TABLE
Plate Stiffness of Absorbent Composite as a Function of Sample
Conditioning

| | Plate Stiffness (N * mm) | | |
|---|---|---|---|
| | as a function of | | Percent |
| | Sample Conditioning | | Reduction at |
| Sample Description | Dried 100° C. | 80% RH | Body |
| 2 pli samples of: | (10 minutes) | (10 minutes) | Conditions |
| Uncoated Spunbond 15 gsm | .23 | .25 | — |
| 42 GSM coating on 15 gsm | 2.12 | 1.44 | 47% |
| spunbond, 0% PEG content | | | |
| 38 GSM coating on 15 gsm | 2.38 | 0.51 | 83% |
| spunbond, 5% PEG content | | | |
| 38 GSM coating on 15 gsm | 1.84 | 0.66 | 91% |
| spunbond, 10% PEG | | | |
| content | | | |
| 41 GSM coating on 15 gsm | 1.88 | 0.31 | 94% |
| spunbond, 15% PEG | | | |
| content | | | |
| 40 GSM coating on 15 gsm | 2.4 | 0.63 | 81% |
| spunbond, 20% PEG | | | |
| content | | | |
| 42 GSM coating on 15 gsm | 2.71 | 0.69 | 80% |
| spunbond, 30% PEG | | | |
| content | | | |

Stiffness of the composites were measured using the “Zwick Flexibility” test. This test is a measure of stiffness of an article as it is deformed downward into a hole beneath the sample. For the test, the sample is modeled as an infinite plate with thickness t that resides on a flat surface where it is centered over a hole with radius R. A central force applied to the foam directly over the center of the hole deflects the foam down into the hole by a distance wwhen loaded in the center by a Force F. For a linear elastic material the deflection can be predicted by:

$$w = \frac{3 F}{4 π E {t}^{3} } ( 1 - v ) ( 3 + v ) {R}^{2}$$

where E is the effective linear elastic modulus, ν is the Poisson's ratio, R is the radius of the hole, and t is the thickness of the foam, taken as the caliper in millimeters measured under a load of about 0.35 kPa, applied by a 7.6 cm diameter Plexiglass platen, with the thickness measured with a Sony U60A Digital Indicator. Taking Poisson's ratio as 0.1 (the solution is not highly sensitive to this parameter, so the inaccuracy due to the assumed value is likely to be minor), we can rewrite the previous equation for w to estimate the effective modulus as a function of the flexibility test results:

$$E \approx \frac{2 {R}^{2}}{3 {t}^{3}} \frac{F}{w}$$

The test results are carried out using an MTS Alliance RT/1 testing machine (MTS Systems Corp., Eden Prairie, Minn.) with a 100 N load cell. As a an absorbent composite at least 6.25 cm by 6.25 cm square sits centered over a hole of radius 17 mm on a support plate, a blunt probe of 3.15 mm radius descends at a speed of 2.54 mm/min. When the probe tip descends to 1 mm below the plane of the support plate, the test is terminated. The maximum slope in grams of force/mm over any 0.5 mm span during the test is recorded (this maximum slope generally occurs at the end of the stroke). The load cell monitors the applied force and the position of the probe tip relative to the plane of the support plate is also monitored. The peak load is recorded, and E is estimated using the above equation.
The bending stiffness per unit width can then be calculated as:

$$S = \frac{E {t}^{3}}{12}$$

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. A translucent absorbent composite comprising a substrate and an absorbent material applied to the substrate, the absorbent material comprises an absorbent prepared from a water soluble ionic polymer comprising about 15 to about 99.9% by mass monoethylenically unsaturated polymer units having at least one functional group, about 0.1 to about 20% by mass ester units selected from the group consisting of acrylate and methacrylate ester units having an alkoxysilane functionality, wherein the absorbent composite has a light transmittance of at least 45%.

2. The translucent absorbent composite of claim 1, wherein the functional group of the absorbent material comprises a functional group selected from the group consisting of a carboxylic acid functional group, a sulphonic acid, a phosphoric acid, mixtures thereof and salts thereof.

3. The translucent absorbent composite of claim 1, wherein the substrate comprises a film material.

4. The translucent absorbent composite of claim 1, wherein the substrate comprises a liquid permeable material.

5. The translucent absorbent composite of claim 4, wherein the liquid-permeable material comprises a nonwoven web.

6. The translucent absorbent composite of claim 1, further comprising an additional layer, wherein the substrate and the absorbent material applied to the substrate are adjacent to the additional layer.

7. The translucent absorbent composite of claim 6, wherein the additional layer comprises a liquid permeable material.

8. The translucent absorbent composite of claim 6, wherein the additional layer comprises a liquid impermeable material.

9. The translucent absorbent composite of claim 6, wherein the additional layer alone has a light transmittance of at least 60%.

10. The translucent absorbent composite of claim 6, wherein the substrate comprises a liquid permeable material and the additional layer comprises a liquid impermeable material.

11. The translucent absorbent composite of claim 10, wherein the substrate comprises a nonwoven web and the additional layer comprises a film.

12. The translucent absorbent composite of claim 11, wherein the nonwoven web and the film contain less than about 1% by weight of filler, pigment, or dye.

13. The translucent absorbent composite of claim 6, further comprising a second additional layer, wherein the substrate and the absorbent material applied to the substrate are positioned between the first additional layer and the second additional layer.

14. The translucent absorbent composite of claim 13, wherein the first additional layer comprises a liquid impermeable material and the second additional layer comprises a liquid permeable material.

15. The translucent absorbent composite of claim 14, wherein the first additional layer and the second additional layer each have a light transmittance of at least 60%.

16. The translucent absorbent composite of claim 15, wherein the absorbent composite has a light transmittance of about 65% to about 79%.

17. The translucent absorbent composite of claim 16, wherein the first additional layer and the second additional layer each have a light transmittance of at least 80%.

18. The translucent absorbent composite of claim 1, wherein the absorbent material further comprise between about 0.1% and about 75% by mass of units selected from the group consisting of polyolefin glycol and polyolefin oxide units.

19. The translucent absorbent composite of claim 18, wherein the absorbent material comprises about 25 to about 90% by mass of monoethylenically unsaturated polymer units, about 0.5 to about 15% by mass polyacrylate ester units having an alkoxysilane functionality, and about 10 to about 60% by mass polymer units selected from the group consisting of polyolefin glycol units, polyolefin oxide units, and combinations thereof.

20. The translucent absorbent composite of claim 1, wherein the absorbent material comprises about 25 to about 90% by mass of monoethylenically unsaturated polymer units, about 0.5 to about 15% by mass polyacrylate ester units having an alkoxysilane functionality, and about 10 to about 60% by mass polymer units selected from the group consisting of polyolefin glycol units, polyolefin oxide units, and combinations thereof.

21. A translucent absorbent composite comprising a backing layer and an absorbent layer, said absorbent layer is positioned adjacent the backing layer, the absorbent layer comprises a substrate and an absorbent material applied to the substrate, the absorbent material comprises an absorbent prepared from a water soluble ionic polymer comprising about 15 to about 99.9% by mass monoethylenically unsaturated polymer units having at least one functional group, about 0.1 to about 20% by mass ester units selected from the group consisting of acrylate and methacrylate ester units having an alkoxysilane functionality, wherein the absorbent layer has a light transmittance of at least 45% and the backing layer has a light transmittance of at least 60%, wherein in the absorbent composite has a minimum light transmittance of at least 45%.

22. The absorbent composite of claim 21, further comprising a liner layer, wherein the liner layer is positioned in the absorbent composite such that the absorbent layer is positioned between the liner layer and the backing layer, wherein the liner layer has a light transmittance of at least 60%.

23. The absorbent composite of claim 22, wherein the absorbent composite has a perimeter region and a central region, the backing layer and the liner layer are each present in both the perimeter region and the central region, and the absorbent layer is positioned between the backing layer and the liner layer only in the central region of the absorbent composite.

24. The absorbent composite of claim 23, wherein the perimeter region of the absorbent composite has a light transmittance of at least 80% and the central region has a light transmittance of between about 65% and about 79%.

25. The absorbent of claim 22, wherein the backing layer comprises a liquid impermeable material and the liner layer comprises a liquid permeable material.

26. The absorbent composite of claim 25, wherein the absorbent composite has a perimeter region and a central region, the backing layer and the liner layer are each present in both the perimeter region and the central region, and the absorbent layer is positioned between the backing layer and the liner layer only in the central region of the absorbent composite.

27. The absorbent composite of claim 26, wherein the perimeter region of the absorbent composite has a light transmittance of at least 60% and the central region has a light transmittance of at least 45%.

28. The absorbent composite of claim 27, wherein the perimeter region of the absorbent composite has a light transmittance of at least 80% and the central region has a light transmittance of between about 65% and about 79%.

29. The absorbent composite of claim 28, wherein the liquid impermeable material of the backing layer comprises a film, the absorbent layer substrate comprises a nonwoven web and the liquid permeable material of the liner layer comprises a nonwoven web.

30. The absorbent composite of claim 29, wherein the liquid impermeable material of the backing layer comprises a polyolefin film, the absorbent layer substrate comprises a spunbond nonwoven web and the liquid permeable of the liner layer comprises a spunbond nonwoven web.

31. The absorbent composite of claim 30, wherein each spunbond nonwoven web has a basis weight of between about 7 gsm and about 100 gsm.

32. The absorbent composite of claim 21, wherein the absorbent composite has an overall thickness of between about 0.25 mm to about 2.0 mm.

33. An absorbent article comprising the absorbent composite of claim 21.

34. A translucent absorbent article comprising a backing layer, an absorbent layer and a bodyside liner, said absorbent layer is positioned between the bodyside liner and the backing layer, the absorbent layer comprises a substrate and an absorbent material applied to the substrate, the absorbent material comprises an absorbent prepared from a water soluble ionic polymer water soluble comprising about 15 to about 99.9% by mass monoethylenically unsaturated polymer units having at least one functional group, about 0.1 to about 20% by mass ester units selected from the group consisting of acrylate and methacrylate ester units having an alkoxysilane functionality, wherein the absorbent layer has a light transmittance of at least 45% and the backing layer and the bodyside liner each have a light transmittance of at least 60%, wherein in the absorbent article has a minimum light transmittance of at least 45%.

35. The absorbent article of claim 34, wherein absorbent article has a perimeter region and a central region, the backing layer and the bodyside liner are each present in both the perimeter region and the central region, and the absorbent layer is positioned between the bodyside liner and the backing layer only in the central region of the absorbent composite.

36. The absorbent article of claim 35, wherein the perimeter region of the absorbent article has a light transmittance of at least 60% and the central region of the absorbent article has a light transmittance of at least 45%.

37. The absorbent article of claim 35, wherein the perimeter region of the absorbent article has a light transmittance of at least 80% and the central region of the absorbent article has a light transmittance of between about 65% and about 79%.

38. The absorbent article of claim 37, wherein the backing layer comprises a film or a breathable nonwoven web, the absorbent layer substrate comprises a nonwoven web and the bodyside liner comprises a nonwoven web.

39. The absorbent article of claim 38, wherein the backing layer comprises a polyolefin film, the absorbent layer substrate comprises a spunbond nonwoven web and the bodyside liner comprises a spunbond nonwoven web.

40. The absorbent article of claim 39, wherein each spunbond nonwoven web has a basis weight of between about 7 gsm and about 100 gsm.

41. The absorbent article of claim 34, wherein the absorbent composite has an overall thickness of between about 0.25 mm to about 5.0 mm.

42. The absorbent article of claim 41, wherein the absorbent composite has an overall thickness of between about 0.25 mm to about 1.5 mm.

43. The absorbent article of claim 34, further comprising a garment attachment adhesive applied to a side of the backing layer opposite the absorbent layer, wherein the garment attachment adhesive has a light transmittance of at least 60%.

44. The absorbent article of claim 43, wherein the garment attachment adhesive has a light transmittance of at least 80%.

45. The absorbent article of claim 43, further comprising a peel strip attached to the garment adhesive, wherein the peel strip has a light transmittance of at least 60%.

46. The absorbent article of claim 43, wherein the peel strip has a light transmittance of at least 80%.

47. The absorbent article of claim 34, further comprising a garment attachment adhesive and a peel strip, said garment attachment adhesive is applied to a side of the backing layer opposite the absorbent layer, wherein the garment attachment adhesive has a light transmittance of at least 80% and the peel strip has a light transmittance of at least 80%, wherein the absorbent article has a perimeter region and a central region, the backing layer and the bodyside liner are each present in both the perimeter region and a central region, the absorbent layer is positioned between the bodyside liner and the backing layer only in the central region of the absorbent article; and the perimeter region of the absorbent article has a light transmittance of at least 70% and the central region of the absorbent article has a light transmittance of between about 65% and about 79%.

48. The absorbent article of claim 34, wherein the absorbent material is impregnated into the substrate of the absorbent layer.

49. The absorbent article of claim 34, wherein the functional group of the absorbent material comprises a functional group selected from the group consisting of a carboxylic acid functional group, a sulphonic acid, a phosphoric acid, mixtures thereof and salts thereof.

50. The absorbent article of claim 34, wherein the absorbent material comprises about 25 to about 90% by mass of monoethylenically unsaturated polymer units, about 0.5 to about 15% by mass polyacrylate ester units having an alkoxysilane functionality, and about 10 to about 60% by mass polymer units selected from the group consisting of polyolefin glycol units, polyolefin oxide units, and combinations thereof.

51. The absorbent article of claim 34, wherein the absorbent material further comprise between about 0.1% and about 75% by mass of units selected from the group consisting of polyolefin glycol and polyolefin oxide units.

52. The absorbent article of claim 34, wherein the backing layer comprises a liquid impermeable material and the bodyside liner comprises a liquid permeable material.

53. The absorbent article of claim 52, wherein the absorbent article comprises a sanitary napkin, an incontinence pad, a pantiliner, a bandage, a bed pad or a furniture pad.

54. The absorbent article of claim 34, wherein the absorbent article comprises a sanitary napkin, an incontinence pad, a pantiliner, a bandage, a bed pad or a furniture pad.

55. The absorbent article of claim 34, wherein the absorbent article has a perimeter region and a central region, wherein the backing layer, absorbent layer and the bodyside liner cover are present in the central region, and the backing layer and optionally the bodyside liner are present in the perimeter region; the absorbent article further comprises an adhesive applied to a portion of the perimeter region of the absorbent article on the side in which the absorbent layer is located; said adhesive has a light transmittance of at least 80%, and the perimeter region of the absorbent article has a light transmittance of at least 80% and the central region of the absorbent article has a light transmittance of between about 65% and about 79%.

56. The absorbent article of claim 55, wherein the backing layer comprises an apertured film.

57. The absorbent article of claim 56, wherein the absorbent article comprises a bandage.

58. The absorbent article of claim 34, wherein the absorbent article comprises a bed pad or a furniture pad.

59. The absorbent composite of claim 21, wherein absorbent composite has a perimeter region and a central region, the backing layer is present in both the perimeter region and the central region, and the absorbent layer is positioned on the backing layer and in the central region of the absorbent composite.

60. The absorbent composite of claim 59, wherein the perimeter region of the absorbent composite has a light transmittance of at least 80% and the central region has a light transmittance of between about 65% and about 79%.

61. An absorbent article comprising a body contacting surface, a surface opposed the body contacting surface, an absorbent core position between the body contacting surface and the surface opposed the body contacting surface, longitudinal edges extending along an edge of absorbent core, and flaps, said flaps extend from the longitudinal edges of the absorbent article and the flaps comprise an absorbent material wherein the flaps each have a light transmittance of at least 45%.

62. The absorbent of claim 61, wherein the absorbent material of the flaps is prepared from a water soluble ionic polymer comprising about 15 to about 99.9% by mass monoethylenically unsaturated polymer units having at least one functional group, about 0.1 to about 20% by mass ester units selected from the group consisting of acrylate and methacrylate ester units having an alkoxysilane functionality.
